(19) United States
(12) Patent Application Publication
(10) Pub. No.: US 2006/0229503 A1
(43) Pub. Date: Oct. 12, 2006

(54) Device for monitoring vital values of persons needing monitoring

(76) Inventor: Gunter Fluegel, Potsdam (DE)

Correspondence Address: KENYON & KENYON LLP, ONE BROADWAY, NEW YORK, NY, 10004, US

(21) Appl. No.: 11/350,475

(22) Filed: Feb. 8, 2006

(30) Foreign Application Priority Data
Feb. 8, 2005 (DE) 10 2005 006 024.2

(51) Int. Cl. A61B 5/00; G08B 1/08; H04Q 7/00

(52) U.S. Cl. 600/300; 340/539.12; 340/539.13

(57) ABSTRACT

A device for monitoring vital values or functions of frail persons or persons needing monitoring is provided. The device includes units for recording and analyzing the vital values, units for determining the location of a person equipped with the device, and a mobile radio transmitter and receiver. When the monitoring function is turned on and at least one critical value is detected in the person needing monitoring, an emergency call number is automatically dialed by the device mentioned last and these data transmitted to the emergency call number. The device may be made up of at least two functional modules of which one accommodates at least the units for recording and analyzing the vital values, and one is designed as mobile telephone suitable also for voice communication. Those functional modules operate as a master-slave system.

### FIELD OF THE INVENTION

The present invention relates to a device for monitoring vital values of frail persons. More specifically, the present invention relates to a device for mobile use designed for senior citizens whose vital or life functions require monitoring because of health-related problems.

### BACKGROUND INFORMATION

Systems, such as, for example, an emergency call button available from hospitals, by which persons who find themselves in an emergency situation can alert others at a remote location to their emergency situation are available.

However, especially due to the increased life expectancy of the population, there are more and more people who do not require constant treatment at a hospital and are largely ambulatory despite certain health-related limitations and who take part in public life virtually without restrictions. For example, persons of advanced age are often subject to health risks which may cause their state of health to suddenly take a serious and unexpected turn for the worse. In many cases, such fluctuations in the state of health and wellbeing are caused by diseases of the heart and circulatory system. For instance, bradyarrhythmia or a sudden rise in the blood pressure may unexpectedly pose a serious challenge for such a person.

Solutions have been developed which allow persons who are frail or whose health is compromised to alert others to a possible emergency situation affecting them, even outside a hospital setting or away from their own home, using mobile radio communication. Such solutions can be found, for example, at www.kretschmer-berlin.com/nottel4.htm. A radio-based emergency phone, for example, is described at that site, which has a special emergency call button upon whose activation an immediate, directly dialed connection to an emergency call number is established. Once the connection has been established, it is used to transmit position data pertaining to the current location of the person using the device. German patent reference DE 198 36 118 A1 appears to disclose a device and system for worldwide emergency rescue via one-button operation. This involves a cellular phone having a considerably reduced functionality and a reduced number of keys for the sake of easy operability. In the event of an emergency situation related to health or caused by an accident, an attack or some other event, this is meant to enable the user to call a worldwide central emergency number by pressing only one key. The illustrated cellular phone does not include functional units which, by themselves, would automatically recognize the signs of an impending turn for the worse in the state of health of a person using the device. Moreover, the solution does not offer the possibility of a prognosis regarding the future state of health. As a result, the solution is basically passive and merely indicates the result of a state, such as: “The person had an infarct.” See, for example, German patent reference DE 197 15 542 A1, re a notebook as data-recording device from whose memory previously input patient data is also able to be transmitted to the called location within the framework of an emergency call with the aid of the cellular phone connected to the memory. See also, for example, the above identified Internet website, which relates to a cellular phone, which the person in a personal emergency can place on his or her chest where ECG data are recorded and transmitted to an emergency call number previously dialed by activating an emergency call button. All previously mentioned approaches require an operation by the person struck by an emergency, i.e., at least the activation of a button. However, such activation may no longer be possible in some instances because the person has lost consciousness, for example.

German patent reference DE 198 48 229 discloses a mobile device for recording and transmitting digitalized medical data in which a GSM unit is integrated. The system is either permanently online for transmission of the recorded medical data or it can be connected online in an event-controlled manner and transmit corresponding medical data to a receiving party by mobile radio communication, utilizing the integrated GSM unit. In view of the fact that many people, for example, older people or those who are frail in some other respect, carry a cellular phone these days, this approach requires the user to take a second GSM unit along in addition. And, the rigid coupling of the units for recording the medical data to the GSM unit in an integrated device considerably restricts its flexibility.

### SUMMARY OF THE INVENTION

The present invention provides an approach that allows frail persons or those whose health is compromised to participate in public life in an unrestricted and mobile manner, and makes it possible to assist them quickly if a health-related emergency situation arises. This approach offers great flexibility in use and high user comfort, the emergency-aid function being basically unnoticeable for the user.

Embodiments of the present invention provide a mobile device and/or method for monitoring vital values of frail persons or persons needing care, having units for recording and analyzing the vital values, units for determining the location of a person equipped with the device, and a mobile radio transmitter and receiver for the transmission of data representing the recorded vital values of the person and his/her location determined with the aid of the units for position determination, to an emergency call number. If the monitoring function is turned on and if the units for recording and analyzing the vital values determine at least one critical vital value of the person needing care, automatic dialing of the emergency call number stored in the device and a transmission of the aforementioned data take place. In a further embodiment, the device is made up of at least two functional modules, which are set apart from one another and of which one accommodates at least the units for recording and analyzing the vital values, and one is embodied as, for example, a mobile telephone or smart phone used at least also for voice communication. These functional modules form a master-slave system in which the mobile telephone or smart phone represents the slave mastered by the functional module having the units for recording and analyzing the vital values via a data and signal connection to which, when a critical vital value is detected, the master transmits data including the vital values and regarding the instantaneous location of the person, as well as control signals, which induce the slave to establish a connection and to transmit the data to the emergency call number.

Embodiments of the present invention provide a device and/or method for monitoring the vital values of frail persons is designed as mobile device and made up of units for recording and analyzing the vital values, units for determining the location of a person equipped with the device, and a mobile radio transmitter and receiver. When the monitoring function is switched on and at least one critical vital value is detected in the aforementioned person, the latter device automatically dials an emergency number and transmits these data thereto, the data representing the recorded vital values of the person and the person's current location ascertained by the units for location determination. In a further embodiment, the device is made up of at least two functional modules of which one accommodates at least the units for recording and analyzing the vital values, and one is designed as mobile phone or smart phone, which basically represents a conventional cellular phone and also may be used at least for voice communication, for example, for sending SMS's or possibly e-mail. The mentioned functional modules form a master-slave system in which the mobile telephone or smart phone constitutes the slave mastered by the functional module having the units for recording and analyzing the vital values via a data and signal connection. If a critical vital value is detected, the master transmits to the slave data including the vital values of the monitored person and data of his/her current location as well as control signals, which induce the slave to establish a connection and to transmit the data to the emergency call number. That is to say, the functional module acting as slave within the meaning of the present invention is basically a conventional cellular phone, whose functionality was simply expanded slightly for the particular intended purpose. To this extent, the examination of the device as master-slave system according to the present invention basically relates only to the case where the monitoring function is turned on. The person equipped with the device according to the present invention may use the mentioned mobile telephone entirely in the usual way for voice communication or for transmitting and receiving SMS's or e-mail. However, if the monitoring function is turned on, the person is made able to turn the monitoring function off and on by him/herself, this cellular phone is mastered or controlled by the other functional module having the units for recording and analyzing the vital values. In practice, this has the effect that, if the latter units determine a critical value of vital values, the cellular phone or the mobile telephone will be triggered to transmit data in connection with the vital values and the person's location, regardless of the wishes of the person provided with the device according to the present invention. Such triggering should take place also when the person in question happens to be talking on the telephone at the time or is receiving a telephone call, so that the currently existing telephone connection or the connection being currently established, will be interrupted immediately and a connection established to the emergency call number stored in the cellular phone by configuration.

The units for recording and analyzing the vital values are, for example, situated on a strap which the person equipped with the device wears on an arm or a leg. As an alternative, it may also be accommodated on a belt or in a vest to be worn by the person. In a further embodiment, the units for determining the location of the person utilizing the device are placed together with the aforementioned units in a functional module jointly formed thereby. In a further embodiment, the units for location determination, which are, for example, units for utilizing the GPS or Galileo positioning system, may be positioned in a separate functional module or in the cellular phone. The latter, for example, may be considered since a number of modern mobile radio telephones are themselves equipped with GPS units these days. However, in view of a use with cellular phones having a variety of different features.

In a further embodiment, one may couple the dialing of the emergency call number to absolute limit values of vital values being exceeded or not achieved, i.e., limit values that are valid regardless of the person. In further embodiments, the functional module having the units for recording and analyzing the vital values may have memory means where values for the normal ranges of the vital values of the individual person equipped with the device are able to be stored by configuration. In further embodiments, the corresponding functional module includes at least means for recording and analyzing the blood pressure and/or the pulse rate of a person wearing the functional module.

The transmission of the control signals and the data including the vital values from master to slave may be carried out in a wire-bound manner. In further embodiments, the data and signal connection between master and slave is provided by an infrared connection or a short-distance radio link according to the Blue Tooth standard, for example, for which appropriate interfaces are provided on the functional modules. In view of the possibility, for example, of turning the functional module having the units for recording and analyzing the vital values on or off with the aid of the cellular phone, the mentioned interfaces may be bidirectional interfaces. In further embodiments having bidirectional interfaces, the functional module may be configured to have the units for recording the vital values via the cellular phone. In further embodiments, with the aid of a corresponding menu, one may adjust the measuring interval at which the vital value(s) is/are to be recorded by the units provided for this purpose. In further embodiments, the configuration may relate to the storing of values for normal ranges of vital values, for example, with regard to a still permissible upper and/or lower blood-pressure value of the person in question, thereby establishing the response behavior of the device.

According to embodiments of the present invention, the mobile phone of the device may be optimized in a special way for comfortable operation by the frail or persons with limited capabilities. In further embodiments, one or a plurality of keys assigned to special functions is set apart from the other keys and larger than the others, the number being limited by a satisfactory compromise between required space and simple operability. For example, one option may include a design that is twice as large as the other keys. In further embodiments, the device is geared toward automatically initiating the alert to an emergency number, i.e., by necessity especially whenever the person to be monitored or the patient itself is no longer able to do so by him/herself, for example, due to loss of consciousness. In further embodiments, in addition to the already mentioned key for turning the monitoring function on and off or for activating and deactivating the units for recording and analyzing the vital values, there may be an additional key for manually initiating a transmission of vital values to a physician or a care facility. In further embodiments, a button may be provided that allows the user to initiate an instantaneous recording of the vital values and their display on a display of the mobile telephone. In further embodiments, keys for direct contacting of the fire department, police or, for instance, a special senior citizen service may be provided in an exposed position on the mobile radio device. In further embodiments, the corresponding calling number as well as the calling number for the automatic transmission of the vital values in a possible emergency are stored in the cellular phone.

In embodiments, for example, for persons with limited vision, the keys of the mobile telephone or smart phone bear especially large lettering or are designed to be tactilely differentiable. For example, the keys may include Braille lettering.

### BRIEF DESCRIPTION OF THE DRAWINGS

FIG. 1 shows an exemplary embodiment of the present invention.

FIG. 2 shows another exemplary embodiment of the present invention.

### DETAILED DESCRIPTION

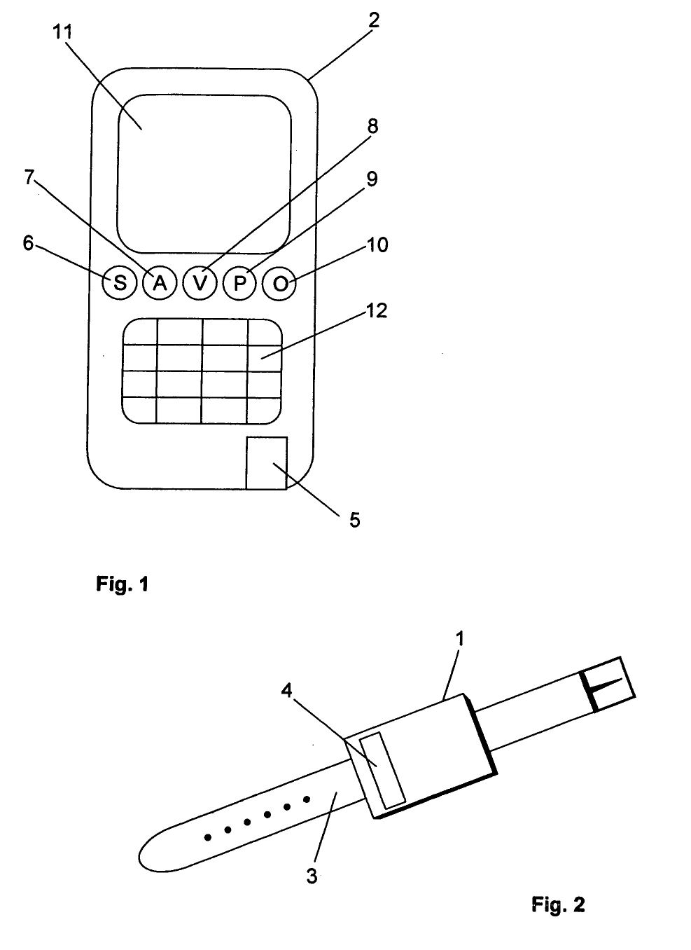

FIGS. 1 and 2 show an exemplary device made up of two functional modules 1, 2. In FIG. 2, The units for recording and analyzing vital values such as the blood pressure or the pulse rate are situated in a module 1 on a strap 3 to be worn on an arm or leg. As an alternative, they may also be accommodated on a belt or in a vest, however this might involve providing cables leading to sensors situated on an arm or leg for the purpose of measuring the blood pressure or pulse. The functional module 1 has at least one transmitter 4 for the transmission of data including the vital values of the person provided with the device, and for the transmission of control signals to cellular phone 2, which has a corresponding receiver 5 for this purpose. In the example shown, a GPS unit also is accommodated in module 1 to be worn on strap 3.

In FIG. 1, second functional module 2 is formed by the cellular phone, which is shown in stylized form in the drawing. This is an available cellular phone 2 according to the GSM or UMTS standard, which may be used for normal voice communication and, as already mentioned, has at least one receiver for the data and control signals transmitted by the functional module 1, and which—as in the example illustrated—for example, has additional, separate function keys 6, 7, 8, 9, 10. With the aid of these function keys 6, 7, 8, 9, 10, the person using the device may manually initiate, among others, a transmission of data of vital values and/or position data to a physician, using key 7, for example. Moreover, keys for contacting a senior service 6 or some other emergency number such as the fire department or police 9 may be provided. In view of preserving the right to autonomy or privacy, an additional function key 8 situated on cellular phone 2 is provided by which the automatic monitoring of the vital values is able to be turned off. This may technically be achieved by either switching receiver 5, provided on cellular phone 2, for data of first functional module 1 having the units for recording and analyzing vital values to inactive, or by switching the just mentioned functional module 1 as shown, for example, in FIG. 2, to inactive. In the latter case, however, the interfaces for the data exchange between functional modules 1, 2 must have bidirectional capability. Even if in the illustrated example the units for detecting and recording the vital values are situated in a shared functional module 1 together with the units for position detection, it is conceivable that they are switchable independently of one another via the cellular phone, by keys 8, 10 in the example. In further embodiments, if GPS is used, the units for position recording need merely include a GPS receiver, if appropriate, an analysis of the GPS data may be carried out only in the emergency call center contacted in an emergency situation.

What is claimed is:

1. A mobile device for monitoring vital values of frail persons, having units for recording and analyzing the vital values, units for determining the location of a person equipped with the device, and a mobile radio transmitter and receiver for the transmission of data representing the recorded vital values of the person and his/her location determined with the aid of the units for position determination, to an emergency call number, whereby, if the monitoring function is turned on and if the units for recording and analyzing the vital values determine at least one critical vital value in said person, automatic dialing of the emergency call number stored in the device and a transmission of the aforementioned data take place,
wherein the device is made up of at least two functional modules, which are set apart from one another and of which one accommodates at least the units for recording and analyzing the vital values, and one is embodied as a mobile telephone or smart phone used at least also for voice communication, and these functional modules form a master-slave system in which the mobile telephone or smart phone represents the slave mastered by the functional module having the units for recording and analyzing the vital values via a data and signal connection to which, when a critical vital value is detected, the master transmits data including the vital values and regarding the instantaneous location of the person, as well as control signals, which induce the slave to establish a connection and to transmit the data to the emergency call number.

2. The device as recited in claim 1, wherein the functional module having the units for recording and analyzing the vital values is situated on a strap to be worn by a person on an arm or a leg, or is situated in a vest the person is to wear.

3. The device as recited in claim 1, wherein the units for location determination are situated together with the units for recording and analyzing the vital values, in a shared functional module.

4. The device as recited in claim 2, wherein the functional module having the units for recording and analyzing the vital values includes memory means in which, via configuration, normal value ranges of the vital values of a person to be equipped with the device are able to be stored.

5. The device as recited in claim 1, wherein the functional module having the units for recording and analyzing the vital values includes at least means for recording and analyzing the blood pressure and/or the pulse rate of a person wearing it.

6. The device as recited in claim 1, wherein the data and signal connection between master and slave is given via infrared interfaces provided thereon.

7. The device as recited in claim 1, wherein the data and signal connection between master and slave is given via a short-distance radio link for which appropriate interfaces are formed on the functional modules.

8. The device as recited in claim 6, wherein the interfaces for data and signal connection of master and slave have a bidirectional design.

9. The device as recited in claim 8, wherein the functional module having the units for recording and analyzing the vital values is able to be configured in a menu-controlled manner via the mobile telephone or smart phone.

10. The device as recited in claim 8, wherein the functional module having the units for recording and analyzing the vital values is able to be activated or deactivated with the aid of at least one operating element situated on the mobile telephone or smart phone.

11. The device as recited in claim 8, wherein an operating element is situated on the mobile telephone or smart phone by whose activation a transmission of vital values to the emergency call number or a doctor may be manually initiated by a person equipped with the device.

12. The device as recited in claim 8, wherein an operating element is situated on the mobile telephone or smart phone by whose activation an immediate recording of the vital values is initiated and these are indicated to the user of the device on a display of the mobile telephone or smart phone independently of a possible transmission to an emergency call number.

13. The device as recited in claim 1, wherein the mobile telephone or smart phone has additional special function keys which are set apart from the other keys of the keyboard and are larger than the remaining keys.

14. The device as recited in claim 12, wherein the special function keys include at least one key for the direct dialing of an emergency call number such as the fire department and police.

15. The device as recited in claim 1, wherein the keys of the mobile telephone or smart phone are designed to be tactilely differentiable.

16. The device as recited in claim 14, wherein the keys of the mobile telephone or smart phone bear Braille lettering.
